(19) United States
(12) Patent Application Publication
(10) Pub. No.: US 2007/0082923 A1
(43) Pub. Date: Apr. 12, 2007

(54) Process for the synthesis of compounds for selectin inhibition

(76) Inventor: Youchu Wang, Sainte-Anne-de-Bellevue (CA)

Correspondence Address: KIRKPATRICK & LOCKHART PRESTON GATES ELLIS LLP, STATE STREET FINANCIAL CENTER, ONE LINCOLN STREET, BOSTON, MA, 02111-2950, US

(21) Appl. No.: 11/543,390

(22) Filed: Oct. 5, 2006

Related U.S. Application Data
(60) Provisional application No. 60/723,734, filed on Oct. 5, 2005.

(51) Int. Cl. C07D 221/06; A61K 31/473

(52) U.S. Cl. 514/290; 546/79

(57) ABSTRACT

The present teachings relate to the field of anti-inflammatory substances, and more particularly to the preparation of compounds that act as antagonists of the mammalian adhesion proteins known as selecting. In some embodiments, the present teachings provide methods for preparing compounds for treating selectin-mediated disorders that have the formula VI: 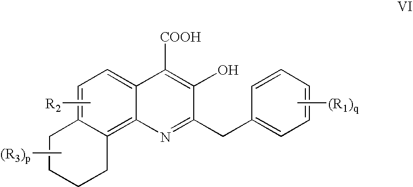
wherein R₁, R₂, R₃, p, and q are defined herein.

This application claims priority to and the benefit of U.S. Provisional Patent Application Ser. No. 60/723,734, filed on Oct. 5, 2005, the entire disclosure of which is incorporated by reference herein.

### FIELD

The present teachings relate to the field of anti-inflammatory substances, and more particularly to the preparation of compounds that act as antagonists of the mammalian adhesion proteins known as selectins.

### BACKGROUND

During the initial phase of vascular inflammation, leukocytes and platelets in flowing blood decrease velocity by adhering to the vascular endothelium and by exhibit rolling behavior. This molecular tethering event is mediated by specific binding of a family of calcium dependent or “C-type” lectins, known as selectins, to ligands on the surface of leukocytes. There are also several disease states that can cause the deleterious triggering of selectin-mediated cellular adhesion, such as autoimmunity disorders, thrombotic disorders, parasitic diseases, and metastatic spread of tumor cells.

The extracellular domain of a selectin protein is characterized by an N-terminal lectin-like domain, an epidermal growth factor-like domain, and varying numbers of short consensus repeats. Three human selectin proteins have been identified, including P-selectin (formerly known as PADGEM or GMP-140), E-selectin (formerly known as ELAM-1), and L-selectin (formerly known as LAM-1). E-selectin expression is induced on endothelial cells by proinflammatory cytokines via its transcriptional activation. L-selectin is constitutively expressed on leukocytes and appears to play a key role in lymphocyte homing. P-selectin is stored in the alpha granules of platelets and the Weibel-Palade bodies of endothelial cells and therefore can be rapidly expressed on the surface of these cell types in response to proinflammatory stimuli. Selectins mediate adhesion through specific interactions with ligand molecules on the surface of leukocytes. Generally the ligands of selectins are comprised, at least in part, of a carbohydrate moiety. For example, E-selectin binds to carbohydrates having the terminal structure: 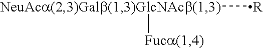
and also to carbohydrates having the terminal structures: 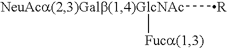
where R is the remainder of the carbohydrate chain. These carbohydrates are known blood group antigens and are commonly referred to as Sialyl Lewis x and Sialyl Lewis a, respectively. The presence of the Sialyl Lewis x antigen alone on the surface of an endothelial cell may be sufficient to promote binding to an E-selectin expressing cell. E-selectin also binds to carbohydrates having the terminal structures:

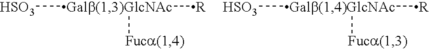

As with E-selectin, each selectin appears to bind to a range of carbohydrates with varying affinities. The strength of the selectin mediated adhesive event (binding affinity) may also depend on the density and context of the selectin on the cell surface.

Structurally diverse glycoprotein ligands, including GlyCAM-1, CD34, ESL-1 and PSGL-1 can bind to selectins with apparent high affinity. PSGL-1 is a mucin-like homodimeric glycoprotein expressed by virtually all subsets of leukocytes and is recognized by each of the three selectins. However PSGL-1 appears to be unique in that it is the predominant high affinity P-selectin ligand on leukocytes. High affinity P-selectin binding to PSGL-1 requires both a SLex containing O-glycan and one or more tyrosine sulfate residues within the anionic N-terminus of the PSGL-1 polypeptide (See Sako, D., et al. Cell 1995; 82(2): 323-331; Pouyani, N., et al., Cell 1995; 82(2): 333-343; Wilkins, P. P., et al., J. Biol. Chem. 1995; 270:39 22677-22680, each of which is incorporated herein by reference in its entirety). L-Selectin also recognizes the N-terminal region of PSGL-1 and has similar sulfation-dependent binding requirements to that of P-selectin. The ligand requirements of E-selectin appear to be less stringent as it can bind to the SLex containing glycans of PSGL-1 and other glycoproteins. Despite the fact that P-selectin knockout and P/E selectin double knockout mice show elevated levels neutrophils in the blood, these mice show an impaired DTH response and delayed thioglycolate induced peritonitis (TIP) response (See Frenette, P. S., et al., Thromb Haemost 1997; 78:1, 60-64, incorporated herein by reference in its entirety). Soluble forms of PSGL-1 such as rPSGL-Ig have shown efficacy in numerous animal models (See Kumar, A., et. al., Circulation. 1999, 99(10) 1363-1369; Takada, M., et. al. J. Clin. Invest 1997, 99(11), 2682-2690; Scalia, R., et al., Circ Res. 1999, 84(1), 93-102, each of which is incorporated herein by reference in its entirety.

In addition, P-selectin ligand proteins, and the gene encoding the same, have been identified. See U.S. Pat. No. 5,840,679, incorporated herein by reference in its entirety. As demonstrated by P-selectin/LDLR deficient mice, inhibition of P-selectin represents a useful target for the treatment of atherosclerosis (See Johnson, R. C., et al., J. Clin. Invest. 1997 99 1037-1043, incorporated herein by reference in its entirety). An increase in P-selectin expression has been reported at the site of atherosclerotic lesions, and the magnitude of the P-selectin expression appears to correlate with the lesion size. It is likely that the adhesion of monocytes, mediated by P-selectin, contributes to atherosclerotic plaque progression (See Molenaar, T. J. M., et al., Biochem. Pharmacol. 2003 (66) 859-866, incorporated herein by reference in its entirety).

Substituted isoquinoline P-selectin inhibitors are disclosed in U.S. patent application Ser. No. 10/984,522, filed Nov. 9, 2004, which is incorporated by reference herein in its entirety for all purposes. Given the role of selectins in numerous important biological processes, including inflammation and adhesion processes, and in disorders such as atherlosclerosis, it can be seen that there is a continuing need for new methods for preparing selectin inhibitors.

### SUMMARY

The present teachings provide methods for the preparation of compounds of formula VI: 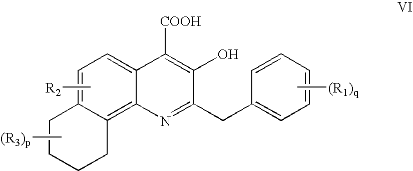
including pharmaceutically acceptable salts, hydrates, and esters thereof, wherein R₁, R₂, R₃, p, and q are as defined herein.

The present teachings also relate to pharmaceutical compositions comprising a compound of formula VI made by the methods disclosed herein, including the pharmaceutically acceptable salts, hydrates and esters of the compound of formula VI.

### DETAILED DESCRIPTION

The present teachings provide methods for the preparation of compounds for antagonizing selectin-mediated intercellular adhesion. In some embodiments, the compounds have the formula VI: 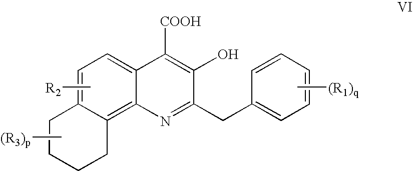
wherein:

p and q are each independently 1, 2 or 3;

each R₁ is independently selected from the group consisting of H, halogen, OH, CN, SH, NH₂, C₁₋₆ alkyl, OC₁₋₆ alkyl, C₁₋₆ perhaloalkyl, OC₁₋₆ perhaloalkyl, C₁₋₆ alkylsulfonamide, C₁₋₆ monoalkylamine, C₁₋₆ dialkylamine, and C₁₋₆ thioalkyl;

R₂ is H, halogen, OH, CN, SH, C₁₋₆ alkyl, OC₁₋₆ alkyl, C₁₋₆ perhaloalkyl, C₁₋₆ thioalkyl, aryl or heteroaryl;
wherein said aryl and said heteroaryl can each optionally be substituted with up to three substituents selected from the group consisting of halogen, OH, CN, SH, NH₂, CHO, CO₂H, NO₂, C₁₋₆ alkyl, OC₁₋₆ alkyl, C₁₋₆ perhaloalkyl and C₁₋₆ thioalkyl; and
wherein said C₁₋₆ alkyl, OC₁₋₆ alkyl and C₁₋₆ thioalkyl can each optionally be substituted with up to three substituents selected from the group consisting of halogen, OH, CN, SH, NH₂, CHO, CO₂H, NO₂, OC₁₋₆ alkyl, C₁₋₆ perhaloalkyl and C₁₋₆ thioalkyl; and

each R₃ is independently selected from the group consisting of H, halogen, OH, CN, SH, NH₂, C₁₋₆ alkyl, OC₁₋₆ alkyl, C₁₋₆ perhaloalkyl, OC₁₋₆ perhaloalkyl, and C₁₋₆ thioalkyl, which compounds include pharmaceutically acceptable salts, hydrates, and esters thereof.

In various embodiments, the method of making a compound of formula VI comprises:

(a) providing compound of formula III: 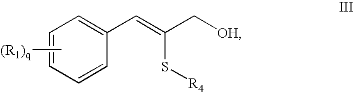
wherein R₁ and q are as defined herein, and R₄ is C₆₋₁₈ alkyl, C₆₋₁₈ alkenyl, C₆₋₁₈ alkynyl, C₆₋₁₀ aryl, C₆₋₁₄ arylalkyl, C₆₋₁₄ alkylaryl, an ether having from about 6 to about 18 carbon atoms, or a polyether having from about 6 to about 24 carbon atoms;

(b) hydrolyzing the compound of formula III to provide a compound of formula IV: 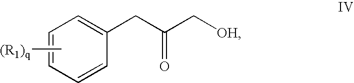
wherein R₁ and q are as defined herein; and

(c) coupling the compound of formula IV with a compound of formula V: 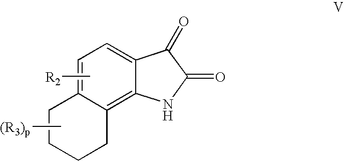
wherein R₂, R₃, and p are as defined herein.

In some embodiments, p is 1; q is 1; and R₂ and R₃ are each H. In some embodiments, p is 1; q is 1; and R₁ is chlorine. In some embodiments, compound III has the structure:

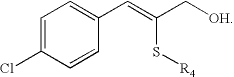

In some embodiments, p is 1; R₂ and R₃ are each H; compound III has the structure:

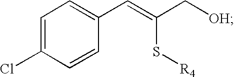

and compound IV has the structure:

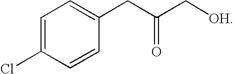

In some embodiments, the compound of formula III is prepared by reaction of a compound of formula II: 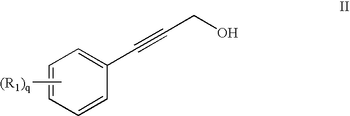
with a thiol compound of formula HS—R₄. For example, in some embodiments, the compound of formula III can be prepared by reaction of a compound of formula HS—R₄ with a compound of formula II wherein q is 1; and R₁ is a chlorine atom attached to the para position of the phenyl ring; i.e., a compound of formula IIa:

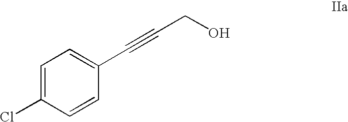

In some embodiments, the compound of formula II is prepared by reaction of a compound of formula I: 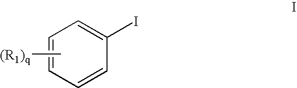
with propargyl alcohol. For example, in some embodiments, the compound of formula II can be prepared by reaction of a compound of formula Ia: 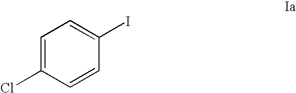
with propargyl alcohol.

In some embodiments, the hydrolyzing of the compound of formula III in step (b) above can be performed in an acidic medium, for example, aqueous sulfuric or hydrochloric acid in methanol.

In some embodiments, the coupling of the compound of formula IV with the compound of formula V in step (c) can be performed in a basic medium, for example, a medium comprising an alcohol and a base, for example, a medium comprising aqueous metal hydroxide, such as sodium hydroxide or potassium hydroxide, and ethanol.

In some embodiments, the reaction of the compound of formula II and HS—R₄ can be performed in a medium comprising a base, for example, a metal hydroxide (e.g., sodium hydroxide or potassium hydroxide), metal methoxide (e.g., sodium methoxide), or metal ethoxide, and an organic solvent, for example, N-methyl pyrrolidinone (NMP), dimethylformamide (DMF), dimethylsulfoxide (DMSO), or dioxane.

In some embodiments, the reaction of the compound of formula I and propargyl alcohol is performed in a medium comprising a metal halide, for example, copper iodide (CuI), and a catalyst, for example, a transition metal catalyst such as a palladium-containing catalyst, for example, Pd/C (with or without PPh₃), PdCl₂(MeCN)₂, PdCl₂(PPh₃)₂, Pd(OAc)₂, PdCl₂, Pd(Ph₃P)₄, and Pd₂dba₃.

In some embodiments, the present teachings provide methods comprising:

(i) reacting a compound of formula I:

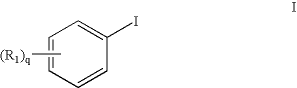

wherein R₁ and q are as defined herein;
with propargyl alcohol to form a compound of formula II:

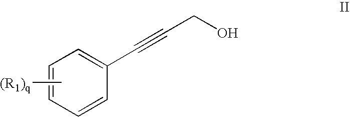

wherein R₁ and q are as defined herein;

(ii) reacting the compound of formula II with a compound of formula HS—R₄, wherein R₄ is as defined herein, to form a compound of formula III:

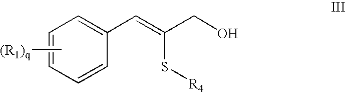

wherein R₁, R₄ and q are as defined herein;

(iii) hydrolyzing the compound of formula III to provide a compound of formula IV:

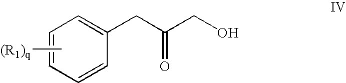

wherein R₁ and q are as defined herein; and

(iv) coupling the compound of formula IV with a compound of formula V:

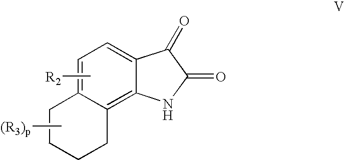

wherein R₂, R₃ and p are as defined herein;
to provide a compound of a compound of formula VI:

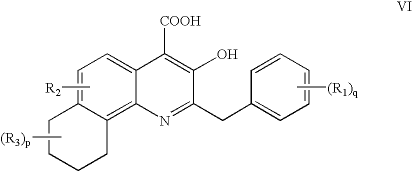

wherein R₁, R₂, R₃, p and q are as defined herein.

In some embodiments, p is 1; q is 1; R₂ and R₃ are each H; and R₁ is chlorine attached to the para position of the phenyl ring.

In some embodiments, step (i) comprises providing a mixture comprising a compound of formula I, a transition metal catalyst, a metal halide, a base and a solvent; and adding propargyl alcohol to the mixture to form the compound of formula II. In some embodiments, the transition metal catalyst can be a palladium-containing catalyst such as PdCl₂(PPh₃)₂, the metal halide can be CuI, and the base can be an amine or inorganic base, such as an alkyl amine (e.g., n-butylamine, triethylamine (Et₃N), N,N-diisopropylethylamine, or piperidine), an aryl amine (e.g., pyridine or 2,6-lutidine) or a metal carbonate (e.g., K₂CO₃). In some embodiments, the solvent can be an ester (e.g., ethyl acetate), an ether (e.g., tetrohydrofuran (THF)), pyridine, N,N,N′,N′-tetramethylethylenediamine (TMEDA), DMSO, or DMF. In some embodiments, the base can be KOH.

In some embodiments, step (ii) comprises providing a mixture comprising a compound of formula II, a solvent, and a base, for example, a metal hydroxide such as sodium hydroxide (NaOH); and adding the compound of formula HS—R₄ to the mixture to form the compound of formula III. In some embodiments, the solvent in step (ii) can be N-methylpyrrolidinone and the metal hydroxide can be NaOH.

In some embodiments, the method further comprises quenching the reaction. In some embodiments, the reaction can be quenched by, for example, addition of water.

In some embodiments, step (iii) comprises providing a mixture comprising a compound of formula III and an alcohol; and adding a protic acid, for example, aqueous sulfuric acid, to the mixture to form the compound of formula IV. In some embodiments, the alcohol in the mixture of step (iii) can be methanol.

In some embodiments, step (iv) comprises providing a mixture comprising a compound of formula V in an aqueous base; heating the mixture; and adding the compound of formula IV to the mixture to form the compound of formula VI.

In some embodiments, each of the methods described herein further includes isolating the compound of formula VI.

In some embodiments of the methods described herein, the compound of formula II, the compound of formula III, or both, are not isolated prior to use in the next reaction.

In some embodiments of the methods described herein, R₄ can be n-dodecyl.

In various embodiments, the compounds of formula VI can be prepared according to general Scheme 1 below.

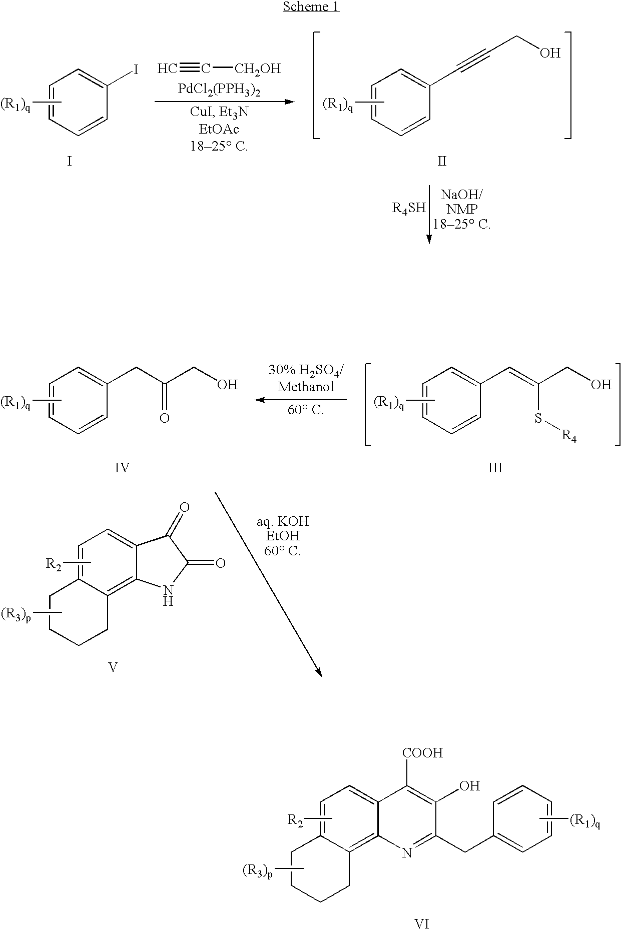

The reaction of compound I with propargyl alcohol can be performed in a solvent using a catalyst, for example, a transition metal catalyst, a metal halide, and a base. Suitable transition metal catalysts include Cu, Ni, Co, Fe, Mn, Cr, V, Ti, Sc, Y, Zr, Nb, Mo, Ru, Rh, Pd, Ag, Cd, La, Hf, Ta, W, Re, Os, Ir, Pt, Au, and Hg, and their donor complexes, for example, phosphine complexes and various salts, hydroxides, oxides, and organometallic derivatives thereof, such as the halides, carboxylates, triflates, tetrafluoroborates, hexafluorophosphates, hexafluoroantimonates, or sulfates and phosphine derivatives thereof, as well as combinations of the foregoing. In some embodiments, the catalyst is a transition metal catalyst, for example a palladium containing catalyst, such as Pd/C (with or without PPh₃), PdCl₂(MeCN)₂, PdCl₂(PPh₃)₂, Pd(OAc)₂, PdCl₂, Pd(Ph₃P)₄ and Pd₂dba₃. In some embodiments, the metal halide is a copper halide, for example, CuI.

Suitable bases for the reaction of compound I with propargyl alcohol include a wide variety of organic and inorganic bases, including but not limited to, trialkylamines such as triethylamine, aromatic bases such as imidazole, N-methylimidazole, pyridine, 2,6-lutidine, 2,4,6-collidine and di-tert-butylpyridines, 4-(dimethylamino)pyridine (DMAP), DBU, DBN, DABCO, N-alkylmorpholines, substituted piperidines, guanidines and anilines, quinoline and substituted quinolines, substituted and unsubstituted pyrrolidines and piperidines, metal hydrides, hydroxides, alkoxides, t-butoxides, oxides, carbonates, and the like. In some embodiments, the base is a trialkylamine, for example, triethylamine.

The reaction of the compound of formula I with propargyl alcohol can be performed at a wide range of temperatures, for example, from about −20° C. to about 250° C. In some embodiments, the reaction is performed at a temperature from about 0° C. to about 50° C., for example, at room temperature (i.e., about 18-25° C.).

A wide variety of solvents can be employed for the reaction as will be apparent to those of skill in the art. For example, suitable solvents include water; alcohols such as methanol (MeOH), ethanol (EtOH), n-propanol, isopropanol, butanols and alkoxyethanols; esters such as ethyl acetate (EtOAc), IPAC and BuOAc; hydrocarbons such as toluene or xylenes; chlorinated hydrocarbons such as dichloromethane, dichloroethane, chloroform, chlorobenzene and ODCB; nitriles such as acetonitrile (CH₃CN), propionitrile, benzonitrile and tolunitrile; ketones such as acetone, MEK, MIBK and cyclohexanone; ethers such as diethyl ether, MTBE, TEHF, DME and DEM; other polar aprotic solvents such as formamide, DMF, DMA, NMP, DMPU, DMSO, and sulfolane or mixtures thereof. In some embodiments, the solvent is an ester, for example, ethyl acetate.

Typically, the compound of formula I is combined with a solvent and a base, and the mixture is then heated, for example, to reflux for about 15 minutes. Subsequently, the mixture can be cooled, for example, to room temperature. The catalyst, for example, PdCl₂(PPh₃)₂, and a metal halide such as CuI then can be added, and after mixing, the propargyl alcohol can be added, for example, while maintaining a low temperature, for example, from about 20° C. to about 30° C. Typically, the reaction mixture is maintained at this low temperature for a period of time, for example, up to about 2 or 3 hours. The compound of formula II then can be isolated, if desired, by any suitable technique. In some embodiments, the reaction mixture is optionally washed, for example, with water, and the mixture containing the compound of formula II is then optionally concentrated and used directly in the next step of the reaction without isolation of the compound of formula II.

Typically, the compound of formula II is reacted with a thiol, for example, of formula R₄SH, where R₄ is as defined herein, in a solvent in the presence of a base to provide a compound of formula III. Suitable bases for the reaction of compound II with the thiol include a wide variety of organic and inorganic bases, including but not limited to, trialkylamines, such as triethylamine, aromatic bases such as imidazole, N-methylimidazole, pyridine, 2,6-lutidine, 2,4,6-collidine and di-tert-butylpyridines, DMAP, DBU, DBN, DABCO, N-alkylmorpholines, substituted piperidines, guanidines and anilines, quinoline and substituted quinolines, substituted and unsubstituted pyrrolidines and piperidines, metal hydrides, hydroxides, alkoxides, t-butoxides, oxides, carbonates, and the like. In some embodiments, the base is a metal hydroxide, for example, sodium hydroxide.

As will be appreciated by those of skill in the art, a variety of solvents are suitable for use in the reaction of the compound of formula II and the thiol. Suitable solvents include, but are not limited to, water; alcohols such as methanol, ethanol, n-propanol, isopropanol, butanols and alkoxyethanols; esters such as EtOAc, IPAc and BuOAc; hydrocarbons such as toluene or xylenes; chlorinated hydrocarbons such as dichloromethane, dichloroethane, chloroform, chlorobenzene and ortho-dichlorobenzene; nitriles such as acetonitrile, propionitrile, benzonitrile and tolunitrile; ketones such as acetone, MEK, MIBK and cyclohexanone; ethers such as diethyl ether, MTBE, THF, DME and DEM; other polar aprotic solvents such as formamide, DMF, DMA, NMP, DMPU, DMSO, and sulfolane or mixtures thereof. In some embodiments, the solvent is an ester, for example, ethyl acetate, or a nitrogen containing organic solvent such as N-methyl pyrrolidinone; or a combination of ethyl acetate and N-methyl pyrrolidinone.

The reaction of the compound of formula II with the thiol can be performed at a wide range of temperatures, for example from about −20° C. to about 250° C. In certain embodiments, the reaction is performed at a temperature from about 0° C. to about 50° C., for example, at room temperature (i.e., about 18-25° C.).

The solution from the preceding reaction containing the compound of formula II is optionally concentrated and a solvent, for example, NMP is added. A compound of formula R₄SH, for example, 1-dodecanethiol, then can be added. After a sufficient time, the reaction can be quenched, for example, by addition of water and a solvent such as ethyl acetate. Subsequently, the compound of formula III can be isolated by any suitable technique if desired. In some embodiments, after quenching, the layers of the reaction mixture are separated and the organic layer can be washed with water, clarified, and diluted with alcohol, for example, methanol. In certain of such embodiments, the resulting solution containing the compound of formula III can be utilized directly in the next step of the reaction without isolation of the compound of formula III.

The hydrolysis of the compound of formula III can be accomplished by a variety of techniques. In some embodiments, the hydrolysis is performed in an acidic medium. A wide variety of acids can be employed in the hydrolysis reaction. Suitable acids include but are not limited to, protic acids such as HCl, HBr, HI, sulfuric acid, phosphoric acid, and carboxylic acids such as acetic acid and trifluoroacetic acid. The acidic medium can further include one or more solvents. Suitable solvents include but are not limited to, water; alcohols such as methanol, ethanol, n-propanol, isopropanol, butanols and alkoxyethanols; esters such as EtOAc, IPAC and BuOAc; hydrocarbons such as toluene or xylenes; chlorinated hydrocarbons such as dichloromethane, dichloroethane, chloroform, chlorobenzene and ODCB; nitrites such as acetonitrile, propionitrile, benzonitrile and tolunitrile; ketones such as acetone, MEK, MIBK and cyclohexanone; ethers such as diethyl ether, MTBE, TEHF, DME and DEM; other polar aprotic solvents such as formamide, DMF, DMA, NMP, DMPU, DMSO, and sulfolane or mixtures thereof. In some embodiments, the solvent is an alcohol such as methanol.

The hydrolysis of the compound of formula III can be performed at a wide range of temperatures, for example from about −20° C. to about 200° C. In certain embodiments, the reaction is performed at a temperature from about 0° C. to about 100° C., for example, at a temperature from about 40° C. to about 80° C., or from about 50° C. to about 70° C., or at about 60° C.

In some embodiments, an aqueous protic acid, for example, 30% sulfuric acid, is added to an alcoholic solution of the compound of formula III, such as the solution described above resulting from the reaction of compound of formula II and the compound of formula R₄SH. The reaction mixture then can be heated, for example, to a temperature of about 60° C., and then cooled, for example, to room temperature. Unreacted thiol can be separated by a suitable technique, for example, extraction with a hydrocarbon solvent. The compound of formula IV can be collected, for example, by concentration of the reaction medium and addition of water to promote crystallization.

The compound of formula VI can be obtained from coupling of the compound of formula IV with the compound of formula V according to a Pfitzinger reaction. Typically, the compound of formula V is heated in an aqueous base. Any base suitable for use in Pfitzinger reactions can be employed. Nonlimiting examples of suitable bases include metal hydroxides such as potassium hydroxide. The coupling reaction can be performed at a temperature greater than about 50° C., for example, at about 90° C., for a sufficient time, for example, about one hour. The reaction mixture then is typically cooled, for example, to about 60° C. The compound of formula IV then can be added, for example, in portions over a period of time, for example, 1-3 hours. The reaction can be quenched, for example, by addition of an acid such as acetic acid, optionally in an organic solvent such as THF. The product can be isolated by any suitable technique.

In various embodiments, in the compounds of formulas I-VI, p is 1; q is 1; R₂ and R₃ are each H; and R₁ is chlorine attached to the para position of the phenyl ring.

Compounds described herein can contain an asymmetric atom (also referred as a chiral center), for example, in an R₁, R₂ and/or R₃ group, and some of the compounds can contain one or more asymmetric atoms or centers, which can thus give rise to optical isomers (enantiomers) and diastereomers. The present teachings and compounds disclosed herein include such optical isomers (enantiomers) and diastereomers (geometric isomers), as well as the racemic and resolved, enantiomerically pure R and S stereoisomers, as well as other mixtures of the R and S stereoisomers and pharmaceutically acceptable salts thereof. Optical isomers can be obtained in pure form by standard procedures known to those skilled in the art, which include, but are not limited to, diastereomeric salt formation, kinetic resolution, and asymmetric synthesis. The present teachings also encompass cis and trans isomers of compounds containing alkenyl moieties (e.g., alkenes and imines). It is also understood that the present teachings encompass all possible regioisomers, and mixtures thereof, which can be obtained in pure form by standard separation procedures known to those skilled in the art, and include, but are not limited to, column chromatography, thin-layer chromatography, and high-performance liquid chromatography.

Ester forms of the present compounds (e.g., compounds where the CO₂H is converted to an ester) include the pharmaceutically acceptable ester forms known in the art including those which can be metabolized into the free acid form, such as a free carboxylic acid, in the animal body, such as the corresponding alkyl esters (e.g., alkyl of 1 to 10 carbon atoms), cyclic alkyl esters, (e.g., of 3-10 carbon atoms), aryl esters (e.g., of 6-20 carbon atoms) and heterocyclic analogues thereof (e.g., of 3-20 ring atoms, 1-3 of which can be selected from oxygen, nitrogen and sulfur heteroatoms) can be used according to the present teachings. The alcoholic residue can carry further substituents. Examples of esters include C₁-C₈ alkyl esters, for example, C₁-C₆ alkyl esters, such as methyl ester, ethyl ester, propyl ester, isopropyl ester, butyl ester, isobutyl ester, t-butyl ester, pentyl ester, isopentyl ester, neopentyl ester, hexyl ester, cyclopropyl ester, cyclopropylmethyl ester, cyclobutyl ester, cyclopentyl ester, and cyclohexyl ester; and aryl esters such as phenyl ester, benzyl ester and tolyl ester.

It is contemplated that the present teachings also include all possible protonated and unprotonated forms of the compounds described herein, as well as solvates, tautomers and pharmaceutically acceptable salts thereof.

More specifically, the methods of the present teachings can be used to prepare compounds of formula VI that can exist as pharmaceutically acceptable salts, including pharmaceutically acceptable acid addition salts prepared from pharmaceutically acceptable acids, including inorganic and organic acids. Such acids include acetic, benzenesulfonic, benzoic, camphorsulfonic, citric, dichloroacetic, ethenesulfonic, formic, fumaric, gluconic, glutamic, hippuric, hydrobromic, hydrochloric, isethionic, lactic, maleic, malic, malonic, mandelic, methanesulfonic, mucic, napthalenesulfonic, nitric, oxalic, pamoic, pantothenic, phosphoric, phthalic, propionic, succinic, sulfuric, tartaric, toluenesulfonic, and as well as other known pharmaceutically acceptable acids. Further representative examples of pharmaceutically acceptable salts can be found in, Journal of Pharmaceutical Science, 66, 2 (1977), incorporated by reference herein for all purposes.

Reacting compounds of this invention with one or more equivalents of an appropriately reactive base may also prepare basic salts. Both mono and polyanionic salts are contemplated, depending on the number of acidic hydrogens available for deprotonation. Appropriate bases can be either organic or inorganic in nature. For example, inorganic bases such as NaHCO₃, Na₂CO₃, KHCO₃, K₂CO₃, Cs₂CO₃, LiOH, NaOH, KOH, NaH₂PO₄, Na₂HPO₄, Na₃PO₄ as well as others are suitable. Organic bases including amines, alkyl amines, dialkylamines, trialkylamines, various cyclic amines (such as pyrrolidine, piperidine, etc) as well as other organic amines are suitable. Quaternary ammonium alkyl salts may also prepared by reacting a compound of the invention with an appropriately reactive organic electrophile (such as methyl iodide or ethyl triflate).

The present teachings also include prodrugs of the compounds described herein. As used herein, “prodrug” refers to a moiety that produces, generates or releases a compound of the present teachings when administered to a mammalian subject. Prodrugs can be prepared by modifying functional groups present in the compounds in such a way that the modifications are cleaved, either by routine manipulation or in vivo, from the parent compounds. Examples of prodrugs include compounds as described herein that contain one or more molecular moieties appended to a hydroxyl, amino, sulfhydryl, or carboxyl group of the compound, and that when administered to a mammalian subject, is cleaved in vivo to form the free hydroxyl, amino, sulfhydryl, or carboxyl group, respectively. Examples of prodrugs can include, but are not limited to, acetate, formate and benzoate derivatives of alcohol and amine functional groups in the compounds of the present teachings. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, the entire disclosures of which are incorporated by reference herein for all purposes.

The compounds described herein can also be administered in the form of liposomes. As is known in the art, liposomes are generally derived from phospholipids or other lipid substances, and are formed by mono or multilamellar hydrated liquid crystals that are dispersed in an aqueous medium. Any nontoxic, pharmacologically acceptable lipid capable of forming liposomes can be used.

The methods disclosed herein can be used to prepare compounds of formula VI or solvates or physiologically functional derivatives thereof, which can be used as active ingredients in pharmaceutical compositions, specifically as selectin inhibitors. The term “selectin inhibitor” is intended to mean a compound that interferes with (i.e., antagonizes) the normal physiological function of selectins in intercellular adhesion.

The present teachings include pharmaceutical compositions comprising at least one compound made by a method described herein and one or more pharmaceutically acceptable carriers, excipients, or diluents. Examples of such carriers are well known to those skilled in the art and can be prepared in accordance with acceptable pharmaceutical procedures, such as, for example, those described in Remington's Pharmaceutical Sciences, 17th edition, ed. Alfonoso R. Gennaro, Mack Publishing Company, Easton, Pa. (1985), the entire disclosure of which is incorporated by reference herein for all purposes. Pharmaceutically acceptable carriers are those that are compatible with the other ingredients in the formulation and are biologically acceptable. Supplementary active ingredients can also be incorporated into the pharmaceutical compositions.

As used herein, the term “alkyl” as a group or part of a group is intended to denote hydrocarbon groups including straight chain, branched and cyclic saturated hydrocarbons. An alkyl group can contain 1-20 carbon atoms. A lower alkyl group can contain up to 4 or up to 6 carbon atoms. A cyclic alkyl group can be monocyclic (e.g., cyclohexyl) or polycyclic (e.g., containing fused, bridged, and/or spiro ring systems), wherein the carbon atoms are located inside or outside of the ring system. Any suitable ring position of a cyclic alkyl group can be covalently linked to the defined chemical structure. Examples of straight chain and branched alkyl groups include methyl (Me), ethyl (Et), propyl (e.g., n-propyl and isopropyl), butyl (e.g., n-butyl, isobutyl, sec-butyl, and t-butyl), pentyl groups (e.g., n-pentyl, isopentyl, and neopentyl), hexyl groups, and the like. Examples of cyclic alkyl groups include cyclopropyl, cyclobutyl, cyclopropylmethyl, cyclopentyl, cyclopentylmethyl, cyclohexyl, cyclohexylmethyl, cyclohexylethyl, and cycloheptyl.

Throughout this specification, it should be understood that the term alkyl is intended to encompass both non-cyclic saturated hydrocarbon groups and cyclic saturated hydrocarbon groups. In some embodiments, alkyl groups are non-cyclic. In other embodiments, alkyl groups are cyclic. In various embodiments, alkyl groups are both cyclic and non-cyclic.

An alkyl group can include one or more halogen substituents, in which case the resulting group can be referred to as a “haloalkyl.” Examples of haloalkyl groups include, but are not limited to, CF₃, C₂F₅, CHF₂, CH₂F, CCl₃, CHCl₂, CH₂Cl, C₂Cl₅, CH₂CF₃, CH₂CH₂CF₂CH₃, CH(CF₃)₂, (CH₂)₆—CF₂CC₃, and the like. “Perhaloalkyl” groups, i.e., alkyl groups wherein all of the hydrogen atoms are replaced with halogen atoms (e.g., CF₃ and C₂F₅), are included within the definition of “haloalkyl” but are also considered an independent subclass of haloalkyls.

As used herein, the term “alkenyl” is intended to denote an alkyl group that contains at least one carbon-carbon double bond. An alkenyl group can contain 2-20 carbon atoms, but typically has a smaller range such as 2-6 carbon atoms. Examples of alkenyl groups include, but are not limited to, ethenyl, propenyl, butenyl, pentenyl, hexenyl, butadienyl, pentadienyl, hexadienyl, vinyl, allyl, 2-methyl-allyl, 4-but-3-enyl, 4-hex-5-enyl, 3-methyl-but-2-enyl, cyclohex-2-enyl, and the like. The one or more carbon-carbon double bonds can be internal (such as in 2-butene) or terminal (such as in 1-butene). Examples of cyclic alkenyl groups include, but are not limited to, cyclopentenyl, cyclohexenyl, cyclohexadienyl, cycloheptatrienyl, and the like.

As used herein, the term “alkynyl” is intended to denote an alkyl group that contains at least one carbon-carbon triple bond. An alkynyl group can contain 2-20 carbon atoms, but typically has a smaller range such as 2-6 carbon atoms. Examples of alkynyl groups include, but are not limited to, ethynyl, propynyl, butynyl such as but-1-yne, pentynyl such as pent-2-yne, ethynyl-cyclohexyl, and the like. The one or more carbon-carbon triple bonds can be internal (such as in 2-butyne) or terminal (such as in 1-butyne).

In some embodiments, alkyl, alkenyl, and alkynyl groups as defined above can be substituted with up to four independently selected substituents. In certain embodiments, these groups are substituted with one, two, or three independently selected substituents. Examples of such substituents include, among others, alkoxy (i.e., O-alkyl, e.g., lower alkoxy, e.g., O—C₁₋₆ alkyl), mono-, di- or trihaloalkoxy (e.g., —O—CX₃ where X is halogen), —(CH₂)ₙNH₂, —(CH₂)ₙNHBoc, C₁₋₆ alkyl, C₁₋₆ perhaloalkyl, OC₁₋₆ alkyl, OC₁₋₆ perhaloalkyl, halogen, thioalkyl, CN, OH, SH, (CH₂)ₙOSO₃H, (CH₂)ₙSO₃H, (CH₂)ₙCO₂R₆, OSO₃R₆, SO₃R₆, SO₂R₆, PO₃R₆R₇, (CH₂)ₙSO₂NR₈R₉, (CH₂)ₙC(═O)NR₈R₉, NR₈R₉, C(═O)R₁₂, aryl, heterocyclo, C(═O)aryl, C(═O)heterocyclo, OC(═O)aryl, OC(═O)heterocyclo, Oaryl, Oheterocyclo, arylalkyl, C(═O)arylalkyl, OC( O)arylalkyl, Oarylalkyl, alkenyl, alkynyl, and NHCOR₈. Other examples of such substituents include phenyl, benzyl, O-phenyl, O-benzyl, —SO₂NH₂, —SO₂NH(C₁₋₆ alkyl), SO₂N(C₁₋₆ alkyl)₂, CH₂COOH, CO₂H, CO₂Me, CO₂Et, CO₂iPr, C(═O)NH₂, C(═O)NH(C₁-C₆), C(═O)N(C₁-C₆)₂, SC₁₋₆ alkyl, OC₁₋₆ alkyl, NO₂, NH₂, CF₃, and OCF₃.

As used herein, the term “alkoxy” is intended to denote an —O-alkyl group. Examples of alkoxy groups include, but are not limited to, methoxy, ethoxy, propoxy (e.g., n-propoxy and isopropoxy), t-butoxy groups, and the like.

As used herein, “thioalkyl” refers to an-S-alkyl group. Examples of thioalkyl groups include, but are not limited to, methylthio, ethylthio, propylthio (e.g., n-propylthio and isopropylthio), t-butylthio groups, and the like.

As used herein, the term “halogen” has its normal meaning of group VII elements, including F, Cl, Br and I.

As used herein, the term “carbocyclic ring” is intended to denote a saturated, partially saturated or aromatic ring system in which the ring atoms are each carbon.

As used herein, the term “aryl” as a group or part of a group is intended to denote an aromatic monocyclic hydrocarbon ring system or a polycyclic ring system where at least one of the rings present in the ring system is an aromatic hydrocarbon ring and any other aromatic rings present in the ring system include only hydrocarbons. In some embodiments, a monocyclic aryl group can have from 6 to 14 carbon atoms and a polycyclic aryl group can have from 8 to 14 carbon atoms. Any suitable ring position of the aryl group can be covalently linked to the defined chemical structure. In some embodiments, an aryl group can have only aromatic carbocyclic rings e.g., phenyl, 1-naphthyl, 2-naphthyl, anthracenyl, phenanthrenyl, pyrenyl groups, and the like. In other embodiments, an aryl group can be a polycyclic ring system in which at least one aromatic carbocyclic ring is fused (i.e., having a bond in common with) to one or more cyclic alkyl or heterocycloalkyl rings. Examples of such aryl groups include, among others, benzo derivatives of cyclopentane (i.e., an indanyl group, which is a 5,6-bicyclic cyclic alkyVaromatic ring system), cyclohexane (i.e., a tetrahydronaphthyl group, which is a 6,6-bicyclic cyclic alkyl/aromatic ring system), imidazoline (i.e., a benzimidazolinyl group, which is a 5,6-bicyclic heterocycloalkyl/aromatic ring system), and pyran (i.e., a chromenyl group, which is a 6,6-bicyclic heterocycloalkyl/aromatic ring system). Other examples of aryl groups include, but are not limited to, benzodioxanyl, benzodioxolyl, chromanyl, indolinyl groups, and the like.

In some embodiments, an aryl group can be substituted with up to 4 independently selected substituents. In certain embodiments, an aryl group is substituted with one, two, or three independently selected substituents. Examples of such substituents include, among others, alkoxy (i.e., O-alkyl, e.g., O-C₁₋₆ alkyl), mono-, di- or trihaloalkoxy (e.g., —O—CX₃ where X is halogen), —(CH₂)ₙNH₂, —(CH₂)ₙNHBoc, C₁₋₆alkyl, C₁₋₆perhaloalkyl, OC₁₋₆alkyl, OC₁₋₆perhaloalkyl, halogen, thioalkyl, CN, OH, SH, (CH₂)ₙOSO₃H, (CH₂)ₙSO₃H, (CH₂)ₙCO₂R₆, OSO₃R₆, SO₃R₆, SO₂R₆, PO₃R₆R₇, (CH₂)ₙSO₂NR₈R₉, (CH₂)ₙC(═O)NR₈R₉, NR₈R₉, C(═O)R₁₂, aryl, heterocyclo, C(═O)aryl, C(═O)heterocyclo, OC(═O)aryl, OC(═O)heterocyclo, Oaryl, Oheterocyclo, arylalkyl, C(═O)arylalkyl, OC(═O)arylalkyl, Oarylalkyl, alkyl such as C₁₋₆ alkyl, alkenyl, alkynyl, and NHCOR₈, wherein the constituent variables are defined herein. Other examples of such substituents include phenyl, benzyl, O-phenyl, O-benzyl, —SO₂NH₂, —SO₂NH(C₁₋₆ alkyl), SO₂N(C₁₋₆ alkyl)₂, CH₂COOH, CO₂H, CO₂Me, CO₂Et, CO₂iPr, C(═O)NH₂, C(═O)NH(C₁-C₆), C(═O)N(C₁-C₆)₂, SC₁₋₆ alkyl, OC₁₋₆ alkyl, NO₂, NH₂, CF₃, and OCF₃.

As used herein, the term “arylalkyl” is intended to denote a group of the formula -alkyl-aryl, wherein aryl and alkyl have the definitions above. In some embodiments, an aryl alkyl group can be substituted with up to 4 independently selected substituents. In certain embodiments, an arylalkyl group is substituted with one, two, or three independently selected substituents. Examples of such substituents include, among others, alkoxy (i.e., O-alkyl, e.g., O—C₁₋₆ alkyl), mono-, di- or trihaloalkoxy (e.g., —O—CX₃ where X is halogen), —(CH₂)ₙNH₂, —(CH₂)ₙNHBoc, C₁₋₆ alkyl, C₁₋₆ perhaloalkyl, OC₁₋₆ alkyl, OC₁₋₆ perhaloalkyl, halogen, thioalkyl, CN, OH, SH, (CH₂)ₙOSO₃H, (CH₂)ₙSO₃H, (CH₂)ₙCO₂R₆, OSO₃R₆, SO₃R₆, SO₂R₆, PO₃R₆R₇, (CH₂)ₙSO₂NR₈R₉, (CH₂)ₙC(═O)NR₈R₉, NR₈R₉, C(═O)R₁₂, aryl, heterocyclo, C(═O)aryl, C(═O)heterocyclo, OC(═O)aryl, OC(═O)heterocyclo, Oaryl, Oheterocyclo, arylalkyl, C(═O)arylalkyl, OC(═O)arylalkyl, Oarylalkyl, alkyl such as C₁₋₆ alkyl, alkenyl, alkynyl, and NHCOR₈, wherein the constituent variables are defined herein. Other examples of such substituents include phenyl, benzyl, O-phenyl, O-benzyl, —SO₂NH₂, —SO₂NH(C₁₋₆ alkyl), SO₂N(C₁₋₆ alkyl)₂, CH₂COOH, CO₂H, CO₂Me, CO₂Et, CO₂iPr, C(═O)NH₂, C(═O)NH(C₁-C₆), C(═O)N(C₁-C₆)₂, SC₁₋₆ alkyl, OC₁₋₆ alkyl, NO₂, NH₂, CF₃, and OCF₃. In some embodiments, the arylalkyl group is a benzyl group that is optionally substituted with 1 to 3 independently selected substituents as described above.

As used herein, “heteroatom” is intended to denote an atom of any element other than carbon or hydrogen and includes, for example, nitrogen, oxygen, sulfur, phosphorus, and selenium.

As used herein, the term “heterocyclo” as a group or part of a group is intended to denote a mono-, bi-, or higher order cyclic ring system that contains at least one ring heteroatom, and optionally contains one or more double or triple bonds. One or more N or S atoms in a heterocyclo can be oxidized (e.g., morpholine N-oxide, thiomorpholine S-oxide, thiomorpholine S,S-dioxide). In some embodiments, nitrogen atoms of heterocycloalkyl groups can bear a substituent as described herein. Heterocyclo groups include fully saturated and partially saturated cyclic heteroatom-containing moieties (containing, e.g., none, or one or more double bonds). Such fully and partially saturated cyclic non-aromatic groups are also collectively referred to herein as “heterocycloalkyl” groups. Heterocycloalkyl groups can also contain one or more oxo groups, such as phthalimide, piperidone, oxazolidinone, pyrimidine-2,4(1H,3H)-dione, pyridin-2(1H)-one, and the like. Examples of heterocycloalkyl groups include, among others, morpholine, thiomorpholine, pyran, imidazolidine, imidazoline, oxazolidine, pyrazolidine, pyrazoline, pyrrolidine, pyrroline, tetrahydrofuran, tetrahydrothiophene, piperidine, piperazine, and the like.

Heterocyclo groups also include cyclic heteroatom-containing moieties that contain at least one aromatic ring. Such fully and partially aromatic moieties are also collectively referred to herein as “heteroaryl” groups. A heteroaryl group, as a whole, can have, for example, from 5 to 13 ring atoms and contain 1-5 ring heteroatoms. Heteroaryl groups include monocyclic heteroaryl rings fused to one or more aromatic carbocyclic rings, non-aromatic carbocyclic rings, and non-aromatic heterocycloalkyl rings. The heteroaryl group can be attached to the defined chemical structure at any heteroatom or carbon atom that results in a stable structure. Generally, heteroaryl rings do not contain O—O, S—S, or S—O bonds. However, one or more N or S atoms in a heteroaryl group can be oxidized (e.g., pyridine N-oxide, thiophene S-oxide, thiophene S,S-dioxide). Examples of heteroaryl groups include, for example, the 5-membered monocyclic and 5-6 bicyclic ring systems shown below:

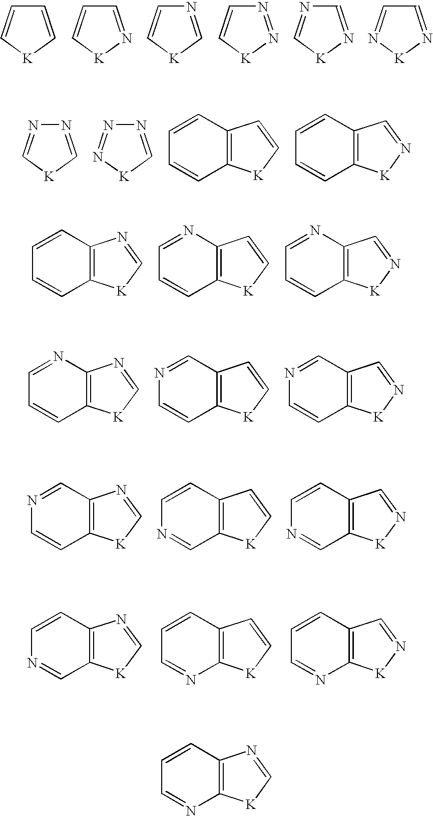

where K is O, S, NH, or NR″, wherein R″ is a substituent described herein that is suitable for a tertiary nitrogen ring atom. Examples of such heteroaryl rings include, but are not limited to, pyrrole, furan, thiophene, pyridine, pyrimidine, pyridazine, pyrazine, triazole, tetrazole, pyrazole, imidazole, isothiazole, thiazole, thiadiazole, isoxazole, oxazole, oxadiazole, indole, isoindole, benzofuran, benzothiophene, quinoline, 2-methylquinoline, isoquinoline, quinoxaline, quinazoline, benzotriazole, benzimidazole, benzothiazole, benzisothiazole, benzisoxazole, benzoxadiazole, benzoxazole, cinnoline, 1H-indazole, 2H-indazole, indolizine, isobenzofuran, naphthyridine, phthalazine, pteridine, purine, oxazolopyridine, thiazolopyridine, imidazopyridine, furopyridine, thienopyridine, pyridopyrimidine, pyridopyrazine, pyridopyridazine, thienothiazole, thienoxazole, and thienoimidazole. Further examples of heteroaryl groups include, but are not limited to, 4,5,6,7-tetrahydroindole, tetrahydroquinoline, benzothienopyridine, benzofuropyridine, and the like. In some embodiments, heteroaryl groups can be substituted with up to four independently selected substituents as described herein.

In some embodiments, heterocyclo groups are:

(a) a five-membered heterocyclic ring containing one to three ring heteroatoms selected from N, S or O exemplified by, but not limited to, furan, imidazole, imidazolidine, isothiazole, isoxazole, oxathiazole, oxazole, oxazoline, pyrazole, pyrazolidine, pyrazoline, pyrrole, pyrrolidine, pyrroline, thiazoline, or thiophene, the five-membered heterocyclic ring being optionally substituted by from 1 to 3 substituents selected from halogen, C₁₋₁₀ alkyl such as C₁₋₆ alkyl, OC₁₋₁₀ alkyl such as OC₁₋₆ alkyl, NO₂, NH₂, CN, CF₃, CO₂H; or

(b) a six-membered heterocyclic ring containing one to three ring heteroatoms selected from N, S or O exemplified by, but not limited to morpholine, oxazine, piperazine, piperidine, pyran, pyrazine, pyridazine, pyridine, pyrimidine, thiadizine, or thiazine, the six-membered heterocyclic ring being optionally substituted by from 1 to 3 substituents selected from halogen, C₁₋₁₀ alkyl, OC₁₋₁₀ alkyl, CHO, CO₂H, NO₂, NH₂, CN, CF₃ or OH; or

(c) a bicyclic ring moiety optionally containing from 1 to 3 ring heteroatoms selected from N or O exemplified by, but not limited to, benzodioxine, benzodioxole, benzofuran, chromene, cinnoline, indazole, indole, indoline, indolizine, isoindole, isoindoline, isoquinoline, napthalene, napthyridine, phthalazine, purine, quinazoline, quinoline, or quinolizine, the bicyclic ring moiety being optionally substituted by from 1 to 3 substituents selected from halogen, C₁₋₆ alkyl, OC₁₋₆ alkyl, CHO, NO₂, NH₂, CN, CF₃, CO₂H, or OH.

As used herein, the term “ether” as group or part of a group is intended to denote the formula —R—O—R′, where R and R′ are each independently selected from alkyl, alkenyl, alkynyl, aryl, arylalkyl and alkylaryl groups as defined above. The term “polyether” is intended to denote compounds comprising the formula —R—(O—R′)ᵥ, where v can be 1 to 10 or higher, and R and each R′ are independently selected from alkyl, alkenyl, alkynyl, aryl, arylalkyl and alkylaryl groups as defined above.

At various places in the present specification, substituents of compounds are disclosed in groups or in ranges. It is specifically intended that the description include each and every individual subcombination of the members of such groups and ranges. For example, the term “C₁₋₁₀ alkyl” is specifically intended to individually disclose C₁, C₂, C₃, C₄, C₅, C₆, C₇, C₈, C₉, C₁₀, C₁-C₁₀, C₁-C₉, C₁-C₈, C₁-C₇, C₁-C₆, C₁-C₅, C₁-C₄, C₁-C₃, C₁-C₂, C₂-C₁₀, C₂-C₉, C₂-C₈, C₂-C₇, C₂-C₆, C₂-C₅, C₂-C₄, C₂-C₃, C₃-C₁₀, C₃-C₉, C₃-C₈, C₃-C₇, C₃-C₆, C₃-C₅, C₃-C₄, C₄-C₁₀, C₄-C₉, C₄-C₈, C₄-C₇, C₄-C₆, C₄-C₅, C₅-C₁₀, C₅-C₉, C₅-C₈, C₅-C₇, C₅-C₆, C₆-C₁₀, C₆-C₉, C₆-C₈, C₆-C₇, C₇-C₁₀, C7-C₉, C₇-C₈, C₈-C₁₀, C₈-C₉, and C₉-C₁₀ alkyl. By way of another example, the term “5-13 membered heteroaryl group” is specifically intended to individually disclose a heteroaryl group having 5, 6, 7, 8, 9, 10, 11, 12, 13, 5-13, 5-12, 5-11, 5-10, 5-9, 5-8, 5-7, 5-6, 6-13, 6-12, 6-11, 6-10, 6-9, 6-8, 6-7, 7-13, 7-12, 7-11, 7-10, 7-9, 7-8, 8-13, 8-12, 8-11, 8-10, 8-9, 9-13, 9-12, 9-11, 9-10, 10-13, 10-12, 10-11, 11-13, 11-12, 12-13 ring atoms.

Throughout the specification, structures may or may not be presented with chemical names. Where any question arises as to nomenclature, the structure prevails.

Throughout the description, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited processing steps.

In the application, where an element or component is said to be included in and/or selected from a list of recited elements or components, it should be understood that the element or component can be any one of the recited elements or components and can be selected from a group consisting of two or more of the recited elements or components.

The use of the singular herein includes the plural (and vice versa) unless specifically stated otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions may be conducted simultaneously.

The compounds of the present teachings can be conveniently prepared in accordance with the procedures outlined in the schemes below, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or specific process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures. Those skilled in the art of organic synthesis will recognize that the nature and order of the synthetic steps presented may be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid chromatography (HPLC) or thin layer chromatography.

Preparation of compounds can involve the protection and deprotection of various chemical groups. The need for protection and deprotection and the selection of appropriate protecting groups can be readily determined by one skilled in the art. The chemistry of protecting groups can be found, for example, in Greene, et al., Protective Groups in Organic Synthesis, 2d. Ed., Wiley & Sons, 1991, the entire disclosure of which is incorporated by reference herein for all purposes.

The reactions of the processes described herein can be carried out in suitable solvents which can be readily selected by one skilled in the art of organic synthesis. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

The following examples illustrate various synthetic routes which can be used to prepare compounds of the present teachings. The following examples are offered for illustrative purposes, and are not intended to limit the invention in any manner. Those of skill in the art will readily recognize a variety of noncritical parameters which can be changed or modified to yield essentially the same results.

### EXAMPLES

HPLC for Examples 1-3 was performed using a Waters 2690 instrument, equipped with a Alltima C₁₈ 3 μm 7×53 mm column. The mobile phase consisted of: Solvent A=H₂O and Solvent B=CH₃CN. Detection was performed by UV at 212 nm. The injection volume of samples was 5 μL. The gradients used are shown in the table below:

| | Gradient | | | |
|---|---|---|---|---|
| | time | Row | % A | % B |
| 1 | | 2.50 | 100.0 | 0.0 |
| 2 | 2.00 | 2.50 | 100.0 | 0.0 |
| 3 | 9.00 | 2.50 | 0.0 | 100.0 |
| 4 | 11.00 | 2.50 | 0.0 | 100.0 |
| 5 | 12.00 | 2.50 | 100.0 | 0.0 |
| 6 | 16.00 | 2.50 | 100.0 | 0.0 |

### Example 1

### Preparation of 3-(4-chlorophenyl)-prop-2-yn-1-ol

To a multi-necked flask equipped with a condenser, a thermometer, an additional funnel and a N₂ inlet was charged 1-chloro-4-iodobenzene (100 g, 0.419 mol), EtOAc (500 mL) and Et₃N (50.9 g, 0.503 mol, 1.2 eq). The mixture was brought to reflux (78.1° C.) for 15 minutes under N₂ and then cooled to 21° C. To the above mixture were charged PdCl₂(PPh₃)₂ (1.47 g, 0.0021 mol, 0.5 mol %) and CuI (0.40 g, 0.0021 mol, 0.5 mol %). The mixture was stirred for 10 minutes. Propargyl alcohol (28.2 g, 0.503 mol) was added dropwise over 15 minutes at a temperature of 20 to 30° C. The reaction was held for 2 hours at 21° C. and was monitored using HPLC as described above. Water (200 mL) was added and the mixture was stirred for 5 min. The aqueous layer was separated as waste. The EtOAc layer, which contained 3-(4-chlorophenyl)-prop-2-yn-1-ol, was washed with water (200 mL) and used directly for the next step. HPLC purity 97%, t_{R} 6.72 min.

### Example 2

### 3-(4-chlorophenyl)-2-dodecylsulfanylprop-2-en-1-ol

The EtOAc solution from Example 1 was concentrated under reduced pressure to 2˜3 volumes, chased with n-methylpyrrolidinone (NMP; 300 mL) and charged with NaOH (18.4 g, 0.46 mol, 1.1 eq based on 1-chloro-4-iodobenzene). To the above mixture was added dropwise 1-dodecanethio (119 g, 0.588 mol, 1.4 eq based on 1-chloro-4-iodobenzene) over 15 minutes at 15-25° C. The reaction was stirred at this temperature for 2 hours. Water (300 mL) and EtOAc (500 mL) were added to quench the reaction and the resulting mixture was stirred for 15 minutes. The EtOAc layer was then separated, washed with water (2×200 mL), and filtered through celite (20 g). The solution was concentrated under reduced pressure, chased with MeOH (250 mL) and finally diluted with MeOH (670 mL) to yield a solution containing 3-(4-chlorophenyl)-2-dodecylsulfanylprop-2-en-1-ol. HPLC purity 97%, t_{R} 11.3 min.

### Example 3

### 1-(4-Chlorophenyl)-3-hydroxypropan-2-one

To the MeOH solution from Example 2, was added dropwise 30% aqueous sulfuric acid (H₂SO₄) (337 mL, 411 g, the ratio of MeOH and 30% H₂SO₄˜2:1) over 20 minutes at 20-30° C. The reaction mixture was heated at 60° C. for 16 hours and cooled to room temperature (20° C.). Heptane (300 mL) was added, and the mixture was stirred for 15 minutes. The heptane layer was separated, and the MeOH layer was extracted with heptane again (300 mL) and concentrated at 40° C. under reduced pressure. Water (700 mL) was added dropwise, and 1-(4-chlorophenyl)-3-hydroxypropan-2-one (48 g, 60% for 3 steps) precipitated out and was air-dried at 21° C. overnight. HPLC purity 97%; mp 53.5° C. ¹H NMR δ (DMSO-d₆, 300 Mz) 7.36 (d, J=8.4 Hz, 2H), 7.21 (d, J=8.4 Hz, 2H), 5.18 (t, J=6.0 Hz, 1H), 4.16 (d, J=6.0 Hz, 2H), 3.77 (s, 2H); ¹³C NMR δ (DMSO-d₆, 75 Hz) 208.8, 134.3, 132.3, 131.9, 128.8, 66.1, 44.3; MS 185 [M+1].

### Example 4

### 2-(4-Chlorobenzyl)-3-hydroxy-7,8,9,10-tetrahydro-benzo[h]quinoline-4-carboxylic acid

To a 100 mL multi-necked flask was charged 6,7,8,9-tetrahydro-1H-benzo[g]indole-2,3-dione (2.5 g, 12.4 mmol, strength 96.2%), followed by addition of aqueous potassium hydroxide (KOH) (3.48 g, 62.1 mmol, in 12 mL H₂O). The suspension was heated to 90° C. for 1 hour, then cooled to 60° C. A solution of 1-(4-chlorophenyl)-3-hydroxypropan-2-one (4.6 g, 24.9 mmol, 2 eq) in THF was added dropwise over 2.5 hours. After 3 hours, the reaction was quenched by addition of THF (10 mL) and acetic acid (4.1 g, 68.3 mmol, 1.1 eq to KOH). The layers were separated and washed with brine (10 mL). Acetic acid (4.1 g, 68.3 mmol, 1.1 eq to KOH) was added to organic layer, and the mixture was heated to 50° C., held for 30 minutes, and then cooled to 21° C. and stirred overnight. Solids were collected and washed with 10% methanol in ethanol (10 mL). The solids were suspended in a mixture of water (20 mL) and 10% methanol in ethanol (5 mL) and stirred for 1 hour, filtered and washed with water (5 mL) to give crude product (5.89 g, wet). The crude product was dissolved in THF/DMF (25 mL) at 68° C. to form a solution. The solution was clarified and added dropwise at 68° C. over 10 minutes with ethanol/H₂O (47 mL) for crystallization, then cooled to 22° C. and stirred for 16 hours. Crystals were collected and dried at 68° C. under vacuum to obtain product (7, 1.76 g, 38.2 % for 2 steps (reaction and purification)). HPLC: (strength: 99.8%, purity: 99.3%); mp 198° C.; ¹H NMR δ (DMSO-d₆, 300 MHz) 8.22 (d, J=8.7 Hz, 1H), 7.33 (m, 4H), 7.27 (d, J=8.7 Hz, 1H), 4.30 (s, 2H), 3.16 (m, 2H), 2.83 (s, 2H), 1.82 (m, 4H); ¹³C δ (DMSO-d₆, 75 MHz) 171.9, 153.0, 151.4, 140.6, 138.3, 135.2, 134.6, 131.6, 131.5, 130.4, 120.8, 123.2, 122.1, 115.6, 39.2, 29.7, 25.3, 23.3, 23.2; MS 368 [M+H].

The HPLC analysis conditions for Example 4 were:

For strength: Waters symmetry, C18, 5 μm, 150×4.6 mm © 30° C., Mobile Phase: 1000 mL H₂O: 0.5 mL H₃PO₄: 200 mL THF: 800 mL CH₃CN: 0.5 mL H₃PO₄.
For purity: ACE, C18, 3 μm, 150×4.6 mm; Mobile Phase A: 400 mg ammonium acetate in 800 mL H₂O: 200 mL CH₃CN Mobile Phase B: 400 mg ammonium acetate in 100 mL H₂O: 900 mL CH₃CN.
Flow rate: 1.0 mL/min; detection at UV 215 nm; injection volume of samples: 10 μL;

detector: WATERS PDA 996; and pump: ALLIANCE SYSTEM F.

GRADIENT:
PURITY GRADIENT

| TIME | % A | % B |
|---|---|---|
| 0 | 100 | 0 |
| 25 | 0 | 100 |
| 35 | 0 | 100 |
| 35.1 | 100 | 0 |
| 50 | 100 | 0 |

It is intended that each of the patents, applications, and printed publications including books mentioned in this patent document be hereby incorporated by reference in their entirety for all purposes.

Variations, modifications, and other implementations of what is described herein will occur to those of ordinary skill in the art without departing from the spirit and the essential characteristics of the present teachings. Accordingly, the scope of the present teachings is to be defined not by the preceding illustrative description but instead by the following claims, and all changes that come within the meaning and range of equivalency of the claims are intended to be embraced herein.

What is claimed is:

1. A method for the preparation of a compound of formula VI: 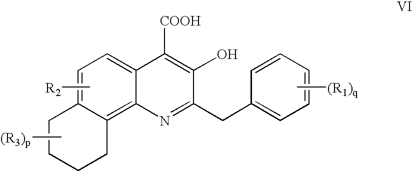
wherein:
each R₁ is independently selected from H, halogen, OH, CN, SH, NH₂, C₁₋₆ alkyl, OC₁₋₆ alkyl, C₁₋₆ perhaloalkyl, C₁₋₆ alkylsulfonamide, C₁₋₆ monoalkylamine, C₁₋₆ dialkylamine, and C₁₋₆ thioalkyl;
R₂ is selected from H, halogen, OH, CN, SH, C₁₋₆ alkyl, OC₁₋₆ alkyl, C₁₋₆ perhaloalkyl, C₁₋₆ thioalkyl, aryl, and heteroaryl;
wherein said aryl and said heteroaryl can each optionally be substituted with up to three substituents selected from halogen, OH, CN, SH, NH₂, C₁₋₆ alkyl, OC₁₋₆ alkyl, C₁₋₆ perhaloalkyl and C₁₋₆ thioalkyl; and
wherein said C₁₋₆ alkyl, OC₁₋₆ alkyl and C₁₋₆ thioalkyl can each optionally be substituted with up to three substituents selected from halogen, OH, CN, SH, NH₂, OC₁₋₆ alkyl, C₁₋₆ perhaloalkyl and C₁₋₆ thioalkyl;
each R₃ is independently selected from H, halogen, OH, CN, SH, NH₂, OC₁₋₆ alkyl, C₁₋₆ perhaloalkyl and C₁₋₆ thioalkyl; and
p and q are each independently 1, 2 or 3;
the method comprising:
hydrolyzing a compound of formula III: 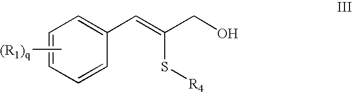
to provide a compound of formula IV: 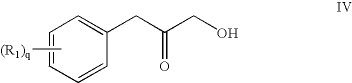
wherein R₁ and q are as defined above; and
R₄ is C₆₋₁₈ alkyl, C₆₋₁₈ alkenyl, C₆₋₁₈ alkynyl, C₆₋₁₀ aryl, C₆₋₁₄ arylalkyl, C₆₋₁₄ alkylaryl, an ether having from about 6 to about 18 carbon atoms, or a polyether having from about 6 to about 24 carbon atoms; and
coupling the compound of formula IV with a compound of formula V: 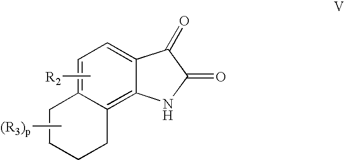
wherein R₂, R₃, and p are as defined above;
to provide a compound of formula VI.

2. The method of claim 1, wherein p is 1; q is 1; and R₂ and R₃ are each H.

3. The method of claim 1, wherein p is 1; q is 1; and R₁ is chlorine.

4. The method of claim 1, wherein compound of formula III has the structure: 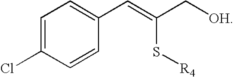
wherein R₄ is as defined above.

5. The method of claim 1, wherein p is 1; R₂ and R₃ are each H; the compound of formula III has the structure: 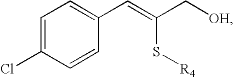
wherein R₄ is as defined above;
and the compound of formula IV has the structure:

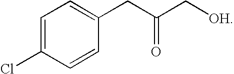

6. The method of claim 5, wherein R₄ is n-dodecyl.

7. The method of claim 1, wherein the compound of formula III is prepared by reacting a compound of formula II: 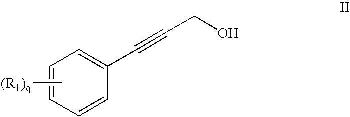
with a compound of formula HS—R₄,
wherein R₁, R₄, and q are as defined above.

8. The method of claim 5, wherein the compound of formula III is prepared by reacting a compound of formula IIa: 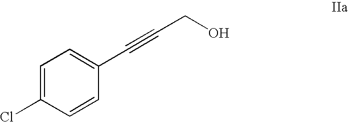
with a compound of formula HS—R₄,
wherein R₄ is as defined above.

9. The method of claim 7, wherein the compound of formula II is prepared by reacting a compound of formula I: 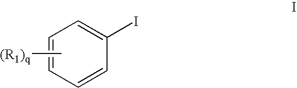
with propargyl alcohol,
wherein R₁ and q are as defined above.

10. The method of claim 8, wherein the compound of formula II is prepared by reacting a compound of formula Ia: 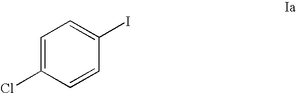
with propargyl alcohol.

11. The method of claim 1, wherein the hydrolyzing is performed in an acidic medium.

12. The method of claim 1, wherein the hydrolyzing is performed in methanolic sulfuric acid.

13. The method of claim 1, wherein the coupling is performed in a medium comprising an alcohol and a base.

14. The method of claim 1, wherein the coupling is performed in a medium comprising aqueous potassium hydroxide and ethanol.

15. The method of claim 7, wherein reacting the compound of formula II with a compound of formula HS—R₄ is performed in a medium comprising a base and an organic solvent.

16. The method of claim 7, wherein reacting the compound of formula II with a compound of formula HS—R₄ is performed in a medium comprising sodium hydroxide and N-methyl pyrrolidinone.

17. The method of claim 9, wherein reacting the compound of formula I with propargyl alcohol is performed in a medium comprising a transition metal catalyst and a metal halide.

18. The method of claim 10, wherein reacting the compound of formula I with propargyl alcohol is performed in a medium comprising a transition metal catalyst and a metal halide.

19. The method of claim 17, wherein the transition metal catalyst is PdCl₂(PPh₃)₂, and the metal halide is CuI.

20. The method of claim 18, wherein the transition metal catalyst is PdCl₂(PPh₃)₂, and the metal halide is CuI.

21. A synthetic method comprising:
(i) reacting a compound of formula I: 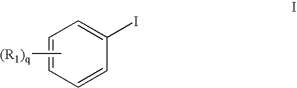
wherein:
q is 1, 2 or 3; and
each R₁ is independently selected from H, halogen, OH, CN, SH, NH₂, C₁₋₆ alkyl, OC₁₋₆ alkyl, C₁₋₆ perhaloalkyl, C₁₋₆ alkylsulfonamide, C₁₋₆ monoalkylamine, C₁₋₆ dialkylamine, and C₁₋₆ thioalkyl;
with propargyl alcohol to form a compound of formula II: 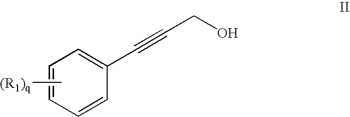
wherein R₁ and q are as defined above;
(ii) reacting the compound of formula 11 with a compound of formula HS—R₄, wherein R₄ is C₆₋₁₈ alkyl, C₆₋₁₈ alkenyl, C₆₋₁₈ alkynyl, C₆₋₁₀ aryl, C₆₋₁₄ arylalkyl, C₆₋₁₄ alkylaryl, an ether having from about 6 to about 18 carbon atoms, or a polyether having from about 6 to about 24 carbon atoms, to form a compound of formula III: 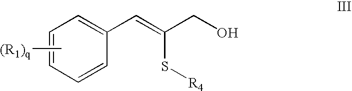
wherein R₁, R₄, and q are as defined above;
(iii) hydrolyzing the compound of formula III to provide a compound of formula IV: 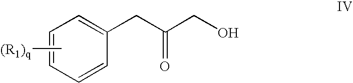
wherein R₁ and q are as defined above and
(iv) coupling the compound of formula IV with a compound of formula V: 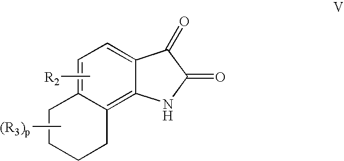
wherein:
p is 1, 2 or 3;
R₂ is H, halogen, OH, CN, SH, C₁₋₆ alkyl, OC₁₋₆ alkyl, C₁₋₆ perhaloalkyl, C₁₋₆ thioalkyl, aryl or heteroaryl;
wherein said aryl and said heteroaryl can each optionally be substituted with up to three substituents selected from the group consisting of halogen, OH, CN, SH, NH₂, C₁₋₆ alkyl, OC₁₋₆ alkyl, C₁₋₆ perhaloalkyl and C₁₋₆ thioalkyl; and
wherein said C₁₋₆ alkyl, OC₁₋₆ alkyl and C₁₋₆ thioalkyl can each optionally be substituted with up to three substituents selected from the group consisting of halogen, OH, CN, SH, NH₂, OC₁₋₆ alkyl, C₁₋₆ perhaloalkyl and C₁₋₆ thioalkyl; and
each R₃ is independently selected from H, halogen, OH, CN, SH, NH₂, OC₁₋₆ alkyl, C₁₋₆ perhaloalkyl and C₁₋₆ thioalkyl;
to provide a compound of formula VI: 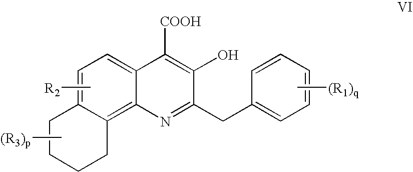
wherein R₁, R₂, R₃, p and q are as defined above.

22. The synthetic method of claim 21, wherein p is 1; q is 1; R₂ and R₃ are each H; and R₁ is chlorine attached to the para position of the phenyl ring.

23. The method of claim 22, wherein step (i) comprises:
providing a mixture comprising a compound of formula I, a transition metal catalyst, a metal halide, a base, and a solvent; and
adding propargyl alcohol to the mixture to form a compound of formula II.

24. The method of claim 23, wherein the transition metal catalyst is PdCl₂(PPh₃)₂, the metal halide is CuI, and the base is a trialkylamine.

25. The method of claim 23, wherein the solvent comprises ethyl acetate.

26. The method of claim 22, wherein step (ii) comprises:
providing a mixture comprising a compound of formula II, a solvent, and a base; and
adding the compound of formula HS—R₄ to the mixture to form the compound of formula III.

27. The method of claim 26, wherein the base is a metal hydroxide.

28. The method of claim 26, wherein the base is NaOH.

29. The method of claim 26, wherein the solvent comprises N-methylpyrrolidinone and the base is NaOH.

30. The method of claim 26, comprising quenching the reaction by addition of water.

31. The method of claim 22, wherein step (iii) comprises:
providing a mixture comprising a compound of formula III and an alcohol; and
adding a protic acid to the mixture to form the compound of formula IV.

32. The method of claim 31, wherein the protic acid is aqueous sulfuric acid.

33. The method of claim 31, wherein the alcohol comprises methanol.

34. The method of claim 22, wherein step (iv) comprises:
providing a mixture comprising a compound of formula V in aqueous base;
heating the mixture; and
adding the compound of formula IV to the mixture to form the compound of formula VI.

35. The method of claim 34, wherein the base is a metal hydroxide.

36. The method of claim 34, wherein the base is KOH.

37. The method of claim 22, comprising isolating the compound of formula VI.

38. The method of claim 9, wherein the compound of formula II is not isolated.

39. The method of claim 10, wherein the compound of formula II is not isolated.

40. The method of claim 7, wherein the compound of formula III is not isolated.

41. The method of claim 8, wherein the compound of formula III is not isolated.

42. The method of claim 9, wherein the compound of formula II and the compound of formula III are not isolated.

43. The method of claim 10, wherein the compound of formula II and the compound of formula III are not isolated.

44. The method of claim 21, wherein the compound of formula II and the compound of formula III are not isolated.

45. The method of claim 22, wherein the compound of formula II and the compound of formula III are not isolated.

46. The method of claim 1, comprising converting the compound of formula VI to a pharmaceutically acceptable salt, hydrate, or ester thereof.

47. The method of claim 21, comprising converting the compound of formula VI to a pharmaceutically acceptable salt, hydrate, or ester thereof.

48. A pharmaceutical composition comprising a compound of formula VI made by the method of claim 1; and a pharmaceutically acceptable carrier or excipient.

49. A pharmaceutical composition comprising a pharmaceutically acceptable salt, hydrate, or ester of a compound of formula VI made by the method of claim 46; and a pharmaceutically acceptable carrier or excipient.
